(19) United States
(12) Patent Application Publication
(10) Pub. No.: US 2024/0200117 A1
(43) Pub. Date: Jun. 20, 2024

(54) Multi-Metabolite Monitoring Biosensor

(71) Applicant: BIOSENSE INC., El Dorado Hills, CA (US)

(72) Inventors: Paul LAMBERT, El Dorado Hills, CA (US); David Shimomoto SANCHEZ, El Dorado Hills, CA (US); Luis Angel Bojorquez VAZQUEZ, El Dorado Hills, CA (US)

(21) Appl. No.: 18/555,853

(22) PCT Filed: Apr. 29, 2021
(86) PCT No.: PCT/US21/29862

§ 371 Date: Oct. 17, 2023

(51) Int. Cl. C12Q 1/00

(52) U.S. Cl. CPC C12Q 1/005; C12Q 1/006

(57) ABSTRACT

A biosensor for monitoring one or more metabolites within a base fluid includes at least one working electrode having an inert conductive layer, a catalyst layer deposited over the inert conductive layer comprising a nanocomposite through which hydrogen peroxide may be adsorbed, an enzyme layer deposited over the catalyst layer comprising one or more enzymes selected to perform a redox reaction towards one or more metabolites, and a binder membrane providing supporting structure to the inert conductive layer, the catalyst layer, and the enzyme layer, the binder membrane selectively allowing molecules having a weight or size below a threshold from passing therethrough. The biosensor also includes an electrical interface in electrical communication with the inert conductive layer and configured to receive an electrical signal indicative of metabolites from the working electrode resulting from a biochemical reaction at or generated by one or more of the layers.

### FIELD

The disclosure relates generally to a non-invasive electrochemical biosensor and biosensing system that can be configured to measure one or more metabolites for various applications.

### BACKGROUND

Biosensors are starting to develop more rapidly because of technological and scientific discoveries. The fast development of solid state, material science and electronic technologies have allowed sensors to be miniaturized and their cost be reduced significantly. Ambulatory sensors are starting to be more affordable to the point of becoming personalized instead of laboratory based. Such advancements have led the field of wearable sensors to emerge as a new market. For example, implantable glucose sensors have become widely used that were not affordable or feasible technologically a few years back.

Dynamic monitoring can be an adjunct to therapy optimization. Underserved populations may greatly benefit from using biosensors especially when they may not have access to hospital/clinic infrastructure. On a broader scale, biosensors have the potential to enhance preventive health programs and improve therapies with feedback that can modulate therapeutic agents or therapy protocols. Global health may improve by increased sensor use to manage health parameters proactively or improve healthcare preventively.

### SUMMARY

In a first aspect, a biosensor for monitoring one or more metabolites within a base fluid is provided The biosensor includes a working electrode having an inert conductive layer, a catalyst layer deposited over the inert conductive layer comprising a nanocomposite through which hydrogen peroxide may be adsorbed, an enzyme layer deposited over the catalyst layer comprising one or more enzymes selected to perform a redox reaction towards one or more metabolites, and a binder membrane providing supporting structure to the inert conductive layer, the catalyst layer, and the enzyme layer, the binder membrane selectively allowing molecules having a weight or size below a threshold from passing therethrough. The biosensor also includes a reference electrode to maintain a known and stable potential, a counter electrode to allow current from or to the working electrode, and an electrical interface in electrical communication with the inert conductive layer and configured to receive an electrical signal from the working electrode resulting from a biochemical reaction at or generated by one or more of the layers. The electrical signal corresponds to a level of the one or more metabolites in the base fluid.

The metabolite can take varying forms including one or more of: glucose, lactate, cholesterol, alcohol, fructose, pyruvate, D-amino acids, Acyl-CoA, or choline.

The base fluid can take varying forms including one or more of sweat, interstitial fluid, mucus, peritoneal fluid, feces, urine, tear fluid, capillary blood, or venous blood.

The one or more enzymes can include lactate oxidase and the one or more metabolites comprise lactate.

The one or more enzymes can include glucose oxidase and the one or more metabolites comprise glucose.

In some variations, the binder membrane can be composed by a selectively permeable porous protection layer on an outermost layer of the working electrode.

In some variation, there are least two electrodes having a corresponding enzyme layer which each comprise a different enzyme which detects a different metabolite.

The binder membrane can include one or more of: chitosan, chitosan deacetylated, fluoropolymer, fluoropolymer-copolymer with the ability of reacting with a cross-linking agent.

The catalyst layer comprises platinum nanoparticles. The catalyst layer can include carbon nanotubes. The carbon nanotubes can be chemically doped with dopant atoms. With such a variation, the dopant atoms can include one or more of nitrogen, boron, fluorine or hydrogen.

The platinum nanoparticles can be physically or chemically bonded to the supporting structure of the binder membrane and walls of the nanotubes.

In another aspect, a biosensor for monitoring one or more metabolites within a base fluid has one or more electrodes. The electrodes include a working electrode having a plurality of layers including (i) a catalyst layer deposited over the inert conductive layer comprising a nanocomposite through which hydrogen peroxide may be adsorbed and (ii) an enzyme layer deposited over the catalyst layer comprising one or more enzymes selected to perform a redox reaction towards one or more metabolites. The biosensor can also include an electrical interface in electrical communication with the one or more electrodes configured to receive an electrical signal resulting from a biochemical reaction at or generated by one or more of the layers, the electrical signal corresponding to a level of the one or more metabolites in the base fluid.

The catalyst layer can include carbon nanotubes which are chemically doped with dopant atoms. The dopant atoms can promote adsorption of molecular oxygen required by the one or more enzymes to perform catalysis of lactate to hydrogen peroxide, and, additionally, promote adsorption of hydrogen peroxide.

In yet another variation, a biosensor for monitoring one or more metabolites within a base fluid includes a plurality of electrodes includes a first working electrode and a second working electrode. The first working electrode has a plurality of layers including a catalyst layer deposited over the inert conductive layer comprising a nanocomposite through which hydrogen peroxide may be adsorbed, and an enzyme layer deposited over the catalyst layer comprising one or more enzymes selected to perform a redox reaction towards a first metabolite. The second working electrode has a plurality of layers including a catalyst layer deposited over the inert conductive layer comprising a nanocomposite through which hydrogen peroxide may be adsorbed and an enzyme layer deposited over the catalyst layer comprising one or more enzymes selected to perform a redox reaction towards a second metabolite different than the first metabolite. The biosensor also includes an electrical interface in electrical communication with the one or more electrodes configured to receive an electrical signal resulting from a biochemical reaction at or generated by one or more of the layers, the electrical signal corresponding to a level of the one or more metabolites in the base fluid.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 is a diagram illustrating aspects of a biosensor;

FIG. 2 is a diagram illustrating an electrode array of a biosensor;

FIG. 3 is a diagram illustrating an exploded view of an example biosensor and housing;

FIG. 4 is a diagram illustrating a first top view of a biosensor;

FIG. 5 is a diagram illustrating a second top view of a biosensor;

FIG. 6 is a first process flow diagram illustrating production of a catalyst nanocomposite;

FIG. 7 is a second process flow diagram illustrating production of a catalyst nanocomposite;

FIG. 8 is a process flow diagram illustrating preparation of an enzyme hybrid nanocomposite-based ink;

FIG. 9 is a first process flow diagram illustrating a modality for disposing layers of a biosensor;

FIG. 10 is a second process flow diagram illustrating a modality for disposing layers of a biosensor;

FIG. 11 is a third process flow diagram illustrating a modality for disposing layers of a biosensor;

FIG. 12 is a process flow diagram illustrating dispensing of nanocomposite, enzyme, and binder;

FIG. 13 is a diagram illustrating laminating procedures for assembling a biosensor;

FIG. 14 is a diagram illustrating layers of a biosensor and a corresponding stacked view;

FIG. 15 is a process flow diagram illustrating a process of detecting a biofluid to initiate electrochemical measurement;

FIG. 16 is a first process flow diagram illustrating a process for initiate reading of a metabolite;

FIG. 17 is a second process flow diagram illustrating a process for initiate reading of a metabolite;

FIG. 18 is a graph illustrating plotted current obtained by amperometry of a biosensor for 60 seconds when in contact with a biofluid conditioned with lactate at a certain concentration; and

FIG. 19 is a diagram illustrating aspects of a computing device for use with a biosensor.

Like reference symbols in the various drawings indicate like elements.

### DETAILED DESCRIPTION

The current subject matter is directed to non-invasive ultra-highly sensitive biosensors which utilizes electrochemical transduction in concert with nanoparticles and enzymes capable of reacting with metabolites. These biosensors can be configured to measure different biological parameters through metabolites which, in turn, can provide important biological information. Biofluids such as saliva, sweat, interstitial fluid or blood contain various metabolites that provide information about the status of an individual metabolism. To accurately monitor such metabolites, the sensitivity of the biosensor is an important factor that determines its usability for a given application. For example, a sensor with higher sensitivity is necessary to detect a specific metabolite in a sample of saliva compared to a sample of blood due to the reduced concentration of metabolites.

The biosensors provided herein can be configured to work with a wide range of metabolites making it versatile for several applications. For example, if the enzyme lactate oxidase is used, the biosensor can detect lactate, if the enzyme glucose oxidase is used, the biosensor can detect glucose.

Concentrations of metabolites in biofluids vary significantly, but there are some patterns that can be noted (Tables 1-2): For lactate, blood and sweat offer similar metabolite concentration ranges, while blood has 11 to 17 times the maximum concentration of Lactate in saliva or interstitial fluid. For glucose, blood has 40 to 80 times metabolite concentration at the minimum concentration range and 7 to 22 times at the maximum concentration range in comparison with saliva, sweat and interstitial fluid.

TABLE 1
Glucose and lactate concentration in a variety of biofluids.

| | METABOLITE | | | |
|---|---|---|---|---|
| | LACTATE (mM) | | GLUCOSE (mM) | |
| | Lower | Maximum | Lower | Maximum |
| | concen- | concen- | concen- | concen- |
| BIOFLUID | tration | tration | tration | tration |
| Blood | 0.8 | 20 | 4 | 11 |
| Saliva | 0.1 | 1.2 | 0.06 | 0.5 |
| Sweat | 2 | 20 *(up to 80 in | 0.1 | 0.8 |
| | | special cases) | | |
| Interstitial fluid | 0.2 | 1.8 | 0.05 | 1.5 |

TABLE 2
Blood metabolite concentration coefficient against other biofluids.

| METABOLITE | |
|---|---|
| LACTATE (times) | GLUCOSE (times) |

| | Lower | Maximum | Lower | Maximum |
|---|---|---|---|---|
| | concen- | concen- | concen- | concen- |
| BIOFLUIDS | tration | tration | tration | tration |
| Blood vs Saliva | 8 | 16.67 | 66.67 | 22 |
| Blood vs Sweat | 0.4 | 1 | 40 | 13.75 |
| Blood vs IF | 4 | 11.11 | 80 | 7.33 |

The biosensor provided herein can incorporate elements that make it substantially more sensitive allowing it to detect millimolar to nanomolar concentration of Lactate and glucose in saliva and sweat.

The biosensor provided herein can be constructed to be ultra-highly sensitive to a specific biosignal making it capable of detecting biological information by measuring small amounts of a plurality of metabolites, such as lactate, glucose and cholesterol present in saliva and/or sweat.

To accomplish detection of the biosignal, a variety of nanotubes, nanoparticles and enzymes can be laid out on the surface of the biosensor with a density that defines the sensitivity of the device. The reaction of the biological sample with the special metabolite in the nanoparticles can generate an electrical signal that is in turn used to quantify the metabolite desired in the biofluid to correlate it with a biological parameter.

The biosensor provided herein is based on the ability to detect hydrogen peroxide as a byproduct of the oxidation of a metabolite carried out by an enzyme. These enzymes belong to the class of the oxidoreductases.

Metabolites compatible with the biosensor can include, for example, lactate, glucose, cholesterol, alcohol, fructose, pyruvate, D-amino acids, acyl-CoA, choline, and others that are common from a clinical perspective.

The working electrode of the biosensor can be configured to read electrical signal associated to the oxidation of a metabolite.

For example, if the metabolite is lactate, the mechanism of oxidation is done by the enzyme lactate oxidase:

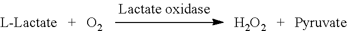

The byproduct of the oxidation of lactate is hydrogen peroxide, which can be detected by the biosensor. Since the stoichiometry of the reaction catalyzed by the enzyme is 1:1, the biosensor can be capable of indirectly quantifying lactate in the biofluid sample.

Due to the high surface area of the nanocomposite catalyst placed on the working electrode, the sensor can adsorb high quantities of hydrogen peroxide due to its affinity towards the embedded donor atoms, e.g., nitrogen, on the nanotube walls.

The working electrode can be protected with a hydrogel, a selectively permeable hydrophobic and/or hydrophilic featured protection layer over the top of the working electrode to avoid the detachment and release of the enzyme or nanomaterials to the biofluid sample.

Other metabolites. The biosensor can be capable of measuring other metabolites based on the detection of hydrogen peroxide. This is done by switching the enzyme on the working electrode, therefore, the biosensor is capable of detecting glucose, cholesterol, alcohol, fructose, pyruvate, D-amino acids, acyl-CoA, choline, etc. The current biosensor can detect a plurality of metabolites in a single sample if necessary, by adding multiple working electrodes.

Other Biofluids. The biosensor can function with several biofluids including saliva, sweat, interstitial fluid, tears, peritoneal fluid, urine, peritoneal fluid, cerebrospinal fluid, mucus, peritoneal fluid, feces, urine, capillary blood, venous blood, etc.

In some cases, the biofluids and/or the biosensor can be preconditioned. For example, if the biosensor is being used to analyze the content of metabolites in saliva, the sample of saliva can be filtered. In some cases, a porous filter can be used to remove any matter/particles above 0.2 μm. The filtering can eliminate bacteria, epithelial death cells, white blood cells, solid particles (detached dental plaque, “food”, or air particles), which in turn, can homogenize saliva eliminating gelling caused by mucin proteins and eliminating bubbles. Filtering saliva conditions it and makes it able to interface into the proposed highly sensitive biosensor. This is carried by using a membrane-based filter, such as PVDF, nylon or cellulose homogenize the biofluid and capture organic and non-organic particles. Other electrochemical process may be used to precondition both the biofluid and biosensor. Saliva filtration increases the ability of the biosensor to derive accurate values of a specific biosignal from the metabolite. The saliva is captured from the patient's mouth using a swab, then, the saliva sampled with the cotton head of the swab is filtered using a membrane-based plunger (e.g., syringeless filter having a membrane).

One example arrangement for characterizing the contents of saliva can start with placing the head of a swab inside the mouth of a subject so that it absorbs saliva from the mouth of the subject. The head of the swab is an absorbent that can be made of cotton or processed cellulose. Depending on the subject, the time to absorb enough saliva varies, but usually 1 min or less is enough. The head can be placed in the supralingual, sublingual and/or buccal region depending on factors relating to the metabolite being analyzed. Once there is sufficient saliva on the head of the swab, the swap can be removed and placed in a filtering chamber (e.g., an opened capsule, a vial, container, etc.).

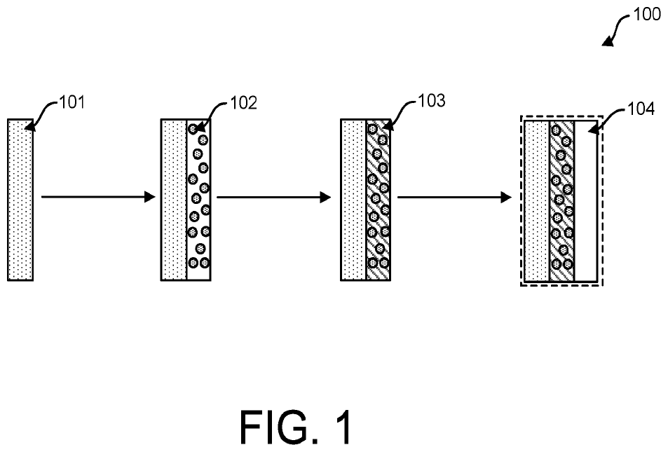

FIG. 1 is a diagram 100 illustrating aspects of a biosensor, and in particular, a working electrode structure 110 that comprises a multi-layer matrix which can, for example, include a binder membrane 101, an enzyme 102, a catalyst (e.g., nanocomposite, etc.) 103, and an inert conductive material 104. The working electrode structure 110 can be configured to allow the biofluids and metabolites to flow across the matrix of layers and interact with the enzyme 102 and catalyst 103. The biochemical reaction from the enzyme 102 (e.g., lactate oxidase, etc.) with the corresponding metabolite (e.g., lactate, etc.) can produce a byproduct (e.g., hydrogen peroxide) that is subsequently adsorbed, processed and transformed into an electrical current signal by the catalyst 103.

The binder membrane 101 can act as a selectively permeable filter/membrane as it blocks high weight biomolecules and allows small water-soluble molecules, such as ions, amino acids and the metabolites of interest (e.g., lactate, etc.), to pass through. The binder membrane 101 can comprise a porous polymer matrix membrane that holds the components of the working electrode together including at least enzyme 102, catalyst 103 and the inert conductive material 104. The binder membrane 101 can be made from a variety of materials including, for example, chitosan, fully or partially deacetylated chitosan, fluoropolymer, and/or fluoropolymer-copolymer with the ability of reacting with a cross-linking agent to increase components entrapment.

The enzyme 102 performs the oxidation of the metabolite of interest, yielding the production of hydrogen peroxide as byproduct. Therefore, the quantification of the metabolite can be achieved indirectly. The enzyme layer including but not limited to one or more of lactate oxidase, glucose oxidase, cholesterol oxidase, alcohol oxidase, fructose oxidase, pyruvate oxidase, D-amino acid oxidase, acyl-CoA oxidase, choline oxidase, etc.

The catalyst 103 (sometimes referred to herein as a nanocomposite) can comprise nanoparticles and nanotubes. The nanoparticles can be physically or chemically bonded to the matrix and wall of the nanotubes. The nanoparticles can have a diameter of up to 100 nm or more which provide a large surface area, an increase the catalytic activity and, inherently, an increase of the sensitivity to hydrogen peroxide. The nanotubes can have an outside diameter of up to 55 nm or more and a length of up to 4 micrometers or more. The nanotubes can be single, double, or multi-walled with the ability to be chemically doped with donor atoms, including but not limited to nitrogen, boron, fluorine, or hydrogen. The doping atoms facilitate adsorption of hydrogen peroxide and the amount of immobilized enzyme in the working electrode, due to the hydrophilicity of the nanocomposite, allowing the enzyme to maintain its bioactivity towards the metabolite. The catalyst 103 can be added on the inert conductive material 104 by one or more deposition techniques.

The inert conductive material 104 can be a pattern that allows for an electrical signal to go from within the biosensor to a computing module which, in some variations, is an external measurement device. The inert conductive material layer 104 can be manufactured using, for example, a number of inert conductive ink formulations through screen-printing technique, roll-to-roll manufacturing, spin coating method, vapor deposition, and other techniques.

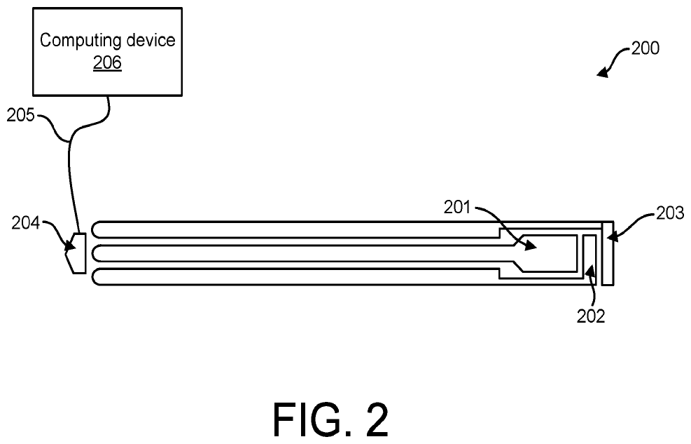

FIG. 2 is a diagram 200 that illustrates an electrode array with a geometry that includes at least one working electrode 201 and one or more of: a counter electrode 202, a reference electrode 203, and/or a detection electrode 204. Other geometries of the electrode design can be utilized depending on the application and/or based on the utilized manufacturing technique. The array of individual electrodes 210 constitutes an electrochemical system that transduces a biochemical reaction into an electrical signal.

To manufacture the biosensor, the individual electrodes can be manufactured by several methods. The working electrode 201 can be made as described above and the other electrodes: counter electrode 202, reference electrode 203, and detection electrode 204 can be printed using ink formulations compatible with screen-printing technique, roll-to-roll manufacturing, spin coating method, vapor deposition, among other techniques.

The working electrode 201 is where the chemical reaction takes place, allowing the biosensor to obtain an electrical signal associated to the concentration of the metabolite of interest as described above. A detection electrode 204 can be provided (e.g., printed, etc.) to allow the module to detect when the biosensor is connected to a remote computing module/device to start measurements. The detection electrode 204 can ground the circuit board 205 (not shown) of the computing device 206 when the biosensor is connected that creates a grounded signal/short. This grounded signal can then drive a transistor switch circuit which can be part of a computing device 206. This signal “switch” is then driven to a processor forming part of the computing device 206 that successfully senses the detection electrode 204 indicating that the biosensor is ready for operation.

The current biosensor can be based on an electrochemical transduction method which utilizes a catalyst nanocomposite that can take the form of an ink as well as other elements. In some variations, the biosensor can take the form of a strip electrode configured by patterns printed or deposited on a plastic substrate along with a capillary chamber, also known as sample chamber, which is the section of the biosensor where the saliva is placed. Since it is composed by hydrophilic films and defined dimensions, the capillary chamber induces capillary action allowing the biofluid (e.g., saliva, etc.) to travel rapidly to make contact with the biosensor electrodes, allowing the biofluid to interact with the biosensing elements 201-204 to later detect the metabolite of interest.

The following description of the fabrication of the biosensor includes two parts: (1) the production of the catalyst nanocomposite, and (2) the manufacturing process of a complete biosensor using other elements.

The biosensor can utilize a catalyst nanocomposite material-based ink, which in combination with the biosensor working electrode 201, an enzyme, and a binder, can detect ultra-low concentrations of hydrogen peroxide. This catalyst nanocomposite, enzyme and binder can be dispensed on the biosensor working electrode 201.

The catalyst nanocomposite can be made of a plurality of different nanomaterials including, as non-limiting examples: (i) mixture of sub-classes of carbon nanotubes not limited to single-walled carbon nanotubes and multi-walled carbon nanotubes doped with donor type atoms: (ii) nanomaterials (nanoparticles, nanotubes, nanoribbons, etc.) that promotes hydrogen peroxide catalysis. The nanomaterials can be composed of metals such as, platinum, nickel, gold, molybdenum, cobalt, or palladium but also from alloys such as oxides and ceramic supported nanomaterials. The nanomaterials can take various shapes including spherical and non-spherical shapes.

In some variations, the biosensor can perform a multi-metabolite reading in the same sample: The biosensor can detect multiple metabolites adding multiple working electrodes that includes different enzyme with a particular catalytic activity against a specific metabolite, such as lactate, glucose, cholesterol, alcohol, fructose, pyruvate, D-amino acids, acyl-CoA and/or acetylcholine.

There can be various configurations of the working electrode 201, counter electrode 202 and reference electrode 203. For multi-metabolite readings, multiple working electrodes can be implemented but the counter electrode and reference electrode can be shared. For these configurations, the electrochemical tests must be done in steps, first the biosensor measures the first metabolite (e.g., glucose), obtains the value, then the biosensor measures the second metabolite (e.g., lactate) and so on, the order does not affect the biosignal detected.

In some cases, there can be multiple cells, each cell comprising a respective working electrode, reference electrode, and counter electrode, which can be configured to detect a different metabolite of interest. For example, a first cell could include a working electrode with the enzyme glucose oxidase for glucose detection and a second cell can include a working electrode with the enzyme lactate oxidase for lactate detection.

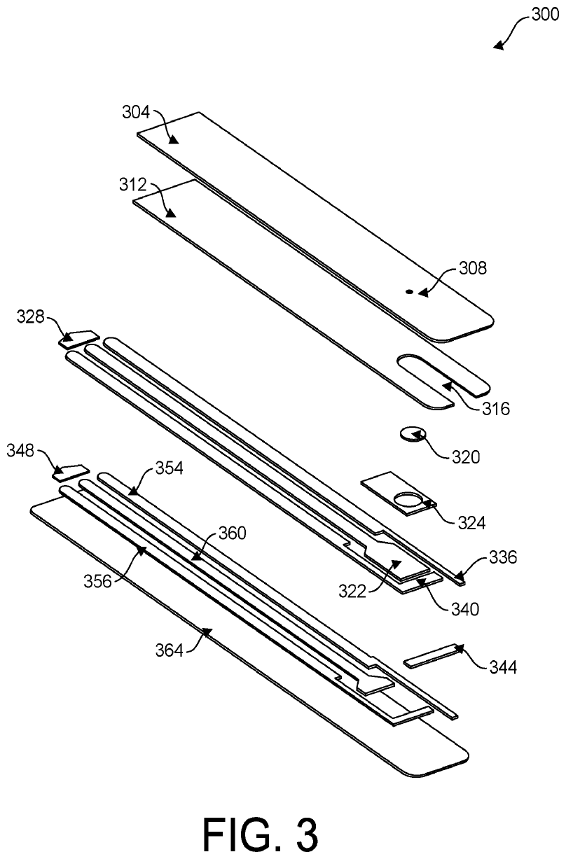

FIG. 3 is a diagram 300 illustrating an exploded view of an example biosensor and housing. The materials and arrangements of FIG. 3 are provided as examples and it will be appreciated that other materials and/or physical configurations can be used. A hydrophilic film 304 can include a vent hole 308. Thereunder, spacer tape 312 can be provided that acts as an intermediate layer. The spacer tape 312 can have a shape and size so as to define a capillary chamber 316 which can comprise capillary material to wick fluids to one or more of the electrodes 201-203. An assemble layer 320 comprising nanocomposite catalyst, enzyme and binder can be disposed over and/or within an inert material layer and surrounded by a dielectric material layer 324. In addition, there can be an inert conductive material 328 for the detection electrode 204, an inert conductive material track working electrode 332, an inert conductive material forming part of the reference electrode 336, and an inert conductive material track 340 for the counter electrode. A silver/silver chloride (Ag/AgCl) layer 344 forming part of the reference electrode. A silver track (e.g., a silver layer) can be configured adjacent to the inert conductive tracks, a silver layer track 348 for the detection electrode, a silver layer track 354 for the counter electrode, a silver layer track for the counter electrode 356, and silver layer track 360 for the working electrode. A substrate 360 is used to dispose (e.g., screen print) the layers of the biosensor and to provide structural support to the other components.

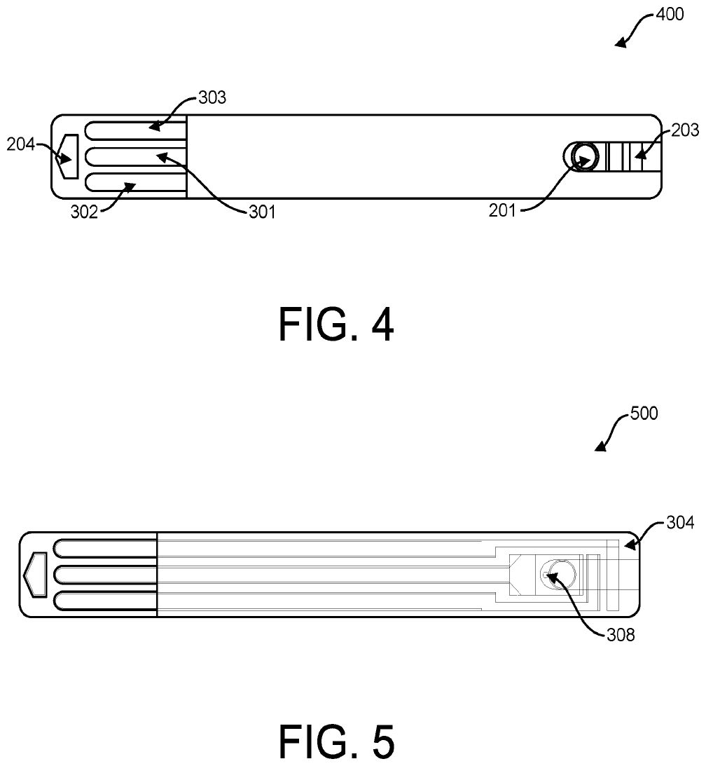

FIG. 4 is a diagram 400 illustrating a top view of a biosensor which includes a connection for the working electrode 301, counter electrode 302, reference electrode 303 and the detection electrode 204. An opening exposes the working electrode 201 and the capillary chamber 316 defines an opening exposing the counter electrode 202 and the reference electrode 203. FIG. 5 is a diagram 500 illustrating another top view of a biosensor illustrating the vent hole 308 in relation to the hydrophilic film 304

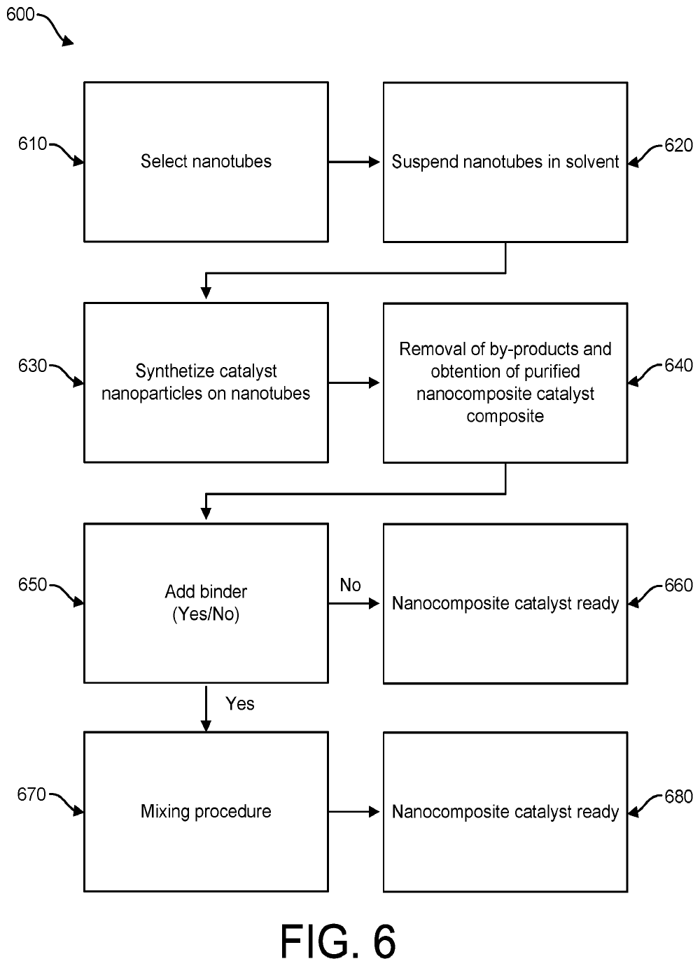

FIG. 6 is a process flow diagram 600 illustration of the production of the catalyst nanocomposite. Initially, at 610, the nanotubes are selected and may vary from single, double, or multi-walled, with the ability to be chemically doped with donor atoms, including but not limited to nitrogen, boron, fluorine, or hydrogen. At 620, a plurality of nanotubes is suspended in a liquid solvent along with the dissolved pre-cursor of the catalyst (usually in the form of a salt) for a later wet synthesis 630. This allows to generate seeds and grow the nanomaterial directly with the nanotubes. The nanocomposite might have nanotubes and nanomaterial that might be chemically or physically bonded between each other. The precursors can take various forms of chemical elements to product the Pt nanoparticles. At 640, the catalyst nanocomposite can be washed using an inert solvent, for example, distilled water. Next, at 650, a binder can be added followed by a mixing procedure 670 resulting in a suspension to acquire the rheological characteristics of a desired catalyst nanocomposite-based ink. Depending on the amount of solvent and binder, the rheology of the ink can change and configured to achieve different viscosity resulting in a ready-to-dispense catalyst ink at 660 and 680.

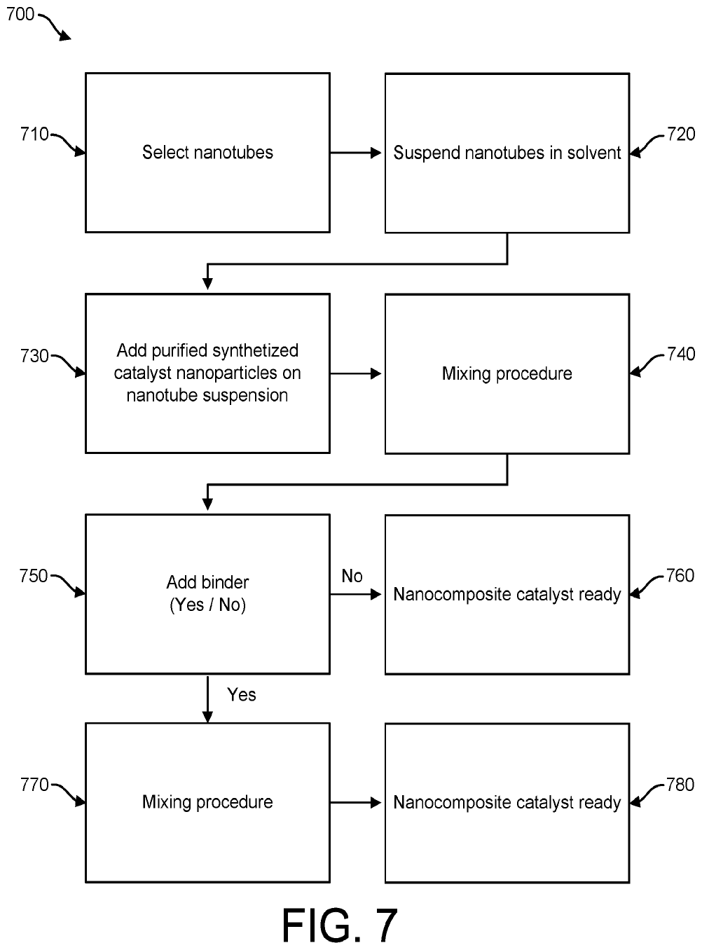

FIG. 7 is a process flow diagram 700 illustration of another modality for producing the catalyst nanocomposite. Initially, at 710, the nanotubes are selected and may vary from single, double, or multi-walled, with the ability to be chemically doped with donor atoms, including but not limited to nitrogen, boron, fluorine, or hydrogen. At 720, a plurality of nanotubes is suspended in a liquid solvent. At 730, the catalyst nanoparticles are added directly (without the wet synthesis) and mixed via an ultrasonic method 740. Finally, at 750, a binder can be added and mixed, at 750, to have an ink that can be dispensed. Depending on the amount of solvent and binder, the rheology of the ink can change and configured to achieve different viscosity. Depending on whether binder is added, the catalyst ink is ready to dispense at 760 and 780.

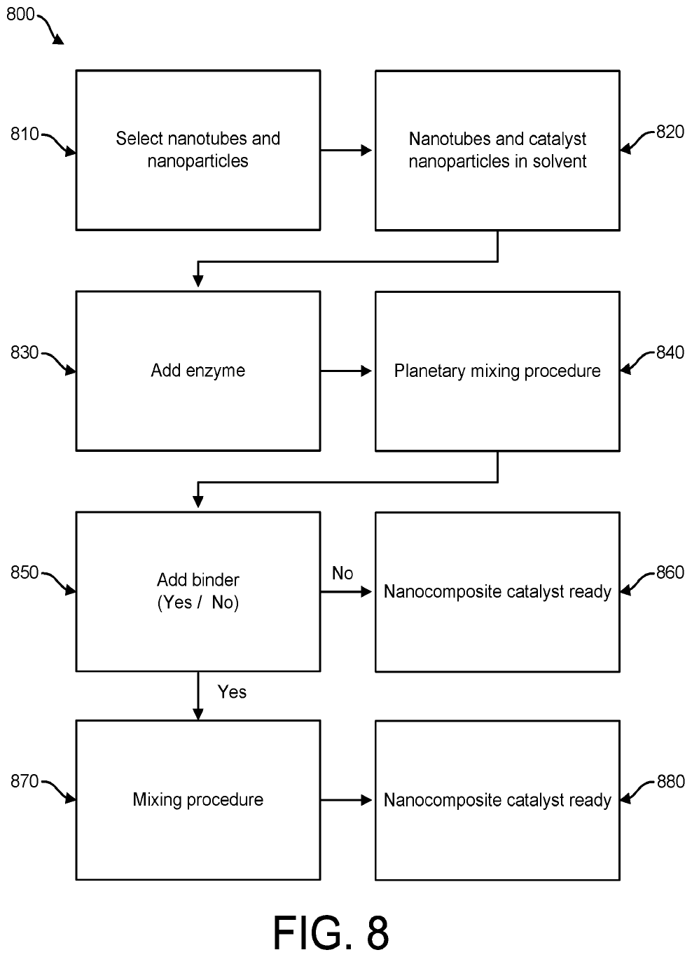

FIG. 8 is a process flow diagram 800 illustration, enzyme hybrid nanocomposite-based ink all-in-one. Initially, at 810, a ratio and type of nanotubes and catalyst are selected. At 820, a purified mixture of a plurality of nanotubes and catalyst nanoparticles on a liquid solvent is ready for the next steps. At 830, an amount of enzyme is added, the enzyme may vary according to the metabolite of interest. At 840, a mixing procedure of enzyme and nanotubes and catalyst nanoparticles is done. The mixing procedure is based on planetary mixing for more than 10 seconds. At 850, binder can be added and mixed, at 850, to the resulting suspension and to acquire the rheological characteristics of a desired catalyst nanocomposite-based ink. Depending on whether the binder is added, the catalyst ink is ready to dispense at 860 and 880.

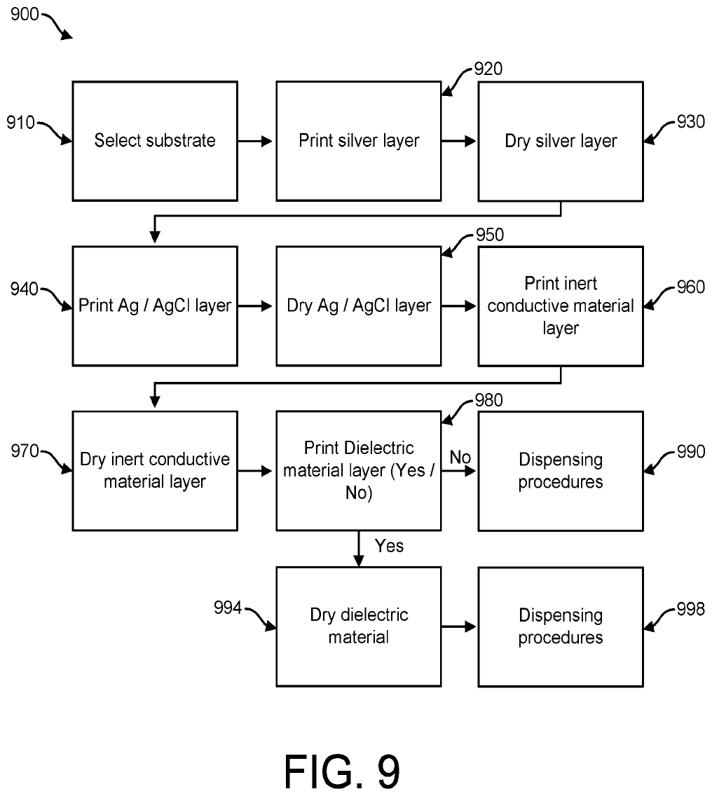

FIG. 9 is a process flow diagram 900 illustration of a modality for disposing the layers of the biosensor. Initially, at 910, the substrate is selected based on terms of inertness and thickness. At 920, a silver layer is printed and then dried at 930. At 940, a silver/silver chloride layer is printed and then dried at 950. At 960 an inert conductive material is printed and then dried at 970. At 970, a dielectric layer is printed to and then dried at 994 to allow the dispensing procedures at 990 or 998.

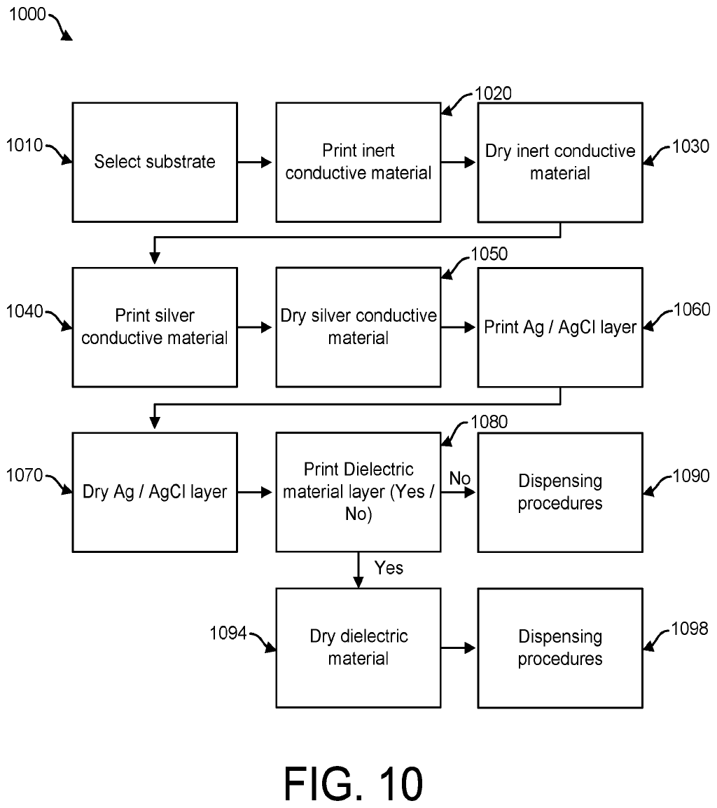

FIG. 10 is a process flow diagram 1000 illustration of a variation of the previous flow diagram. Initially at 1010, a substrate is selected based on terms of inertness and thickness. At 1020, an inert conductive material is printed and then dried at 1030. At 1040, a conductive silver material layer is printed and then dried at 1050. At 1060, a silver/silver chloride layer is printed and then dried at 1070. At 1080. A dielectric layer can be printed to later be dried at 194 to allow the dispensing procedures at 1090 or 1098.

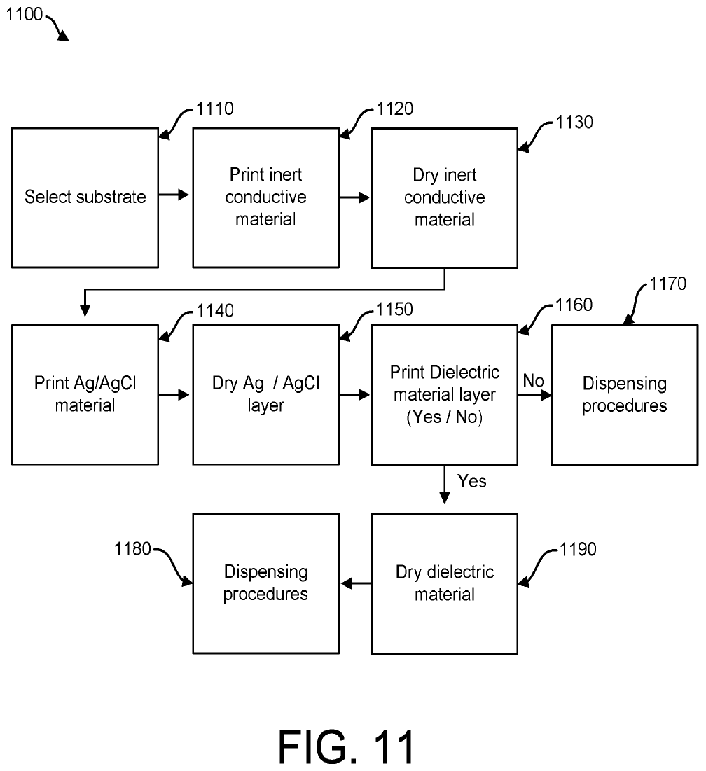

FIG. 11 is a process flow diagram 1100 illustrating a modality for disposing the layers of the biosensor without the silver conductive material. Initially at 1110, a substrate is selected based on terms of inertness and thickness. At 1120, an inert conductive material is printed and then dried at 1130. At 1140, a silver/silver chloride layer is printed and then dried at 1150. At 1160 a dielectric layer can be printed to later be dried at 1180 to allow the dispensing procedures at 1170 and 1180.

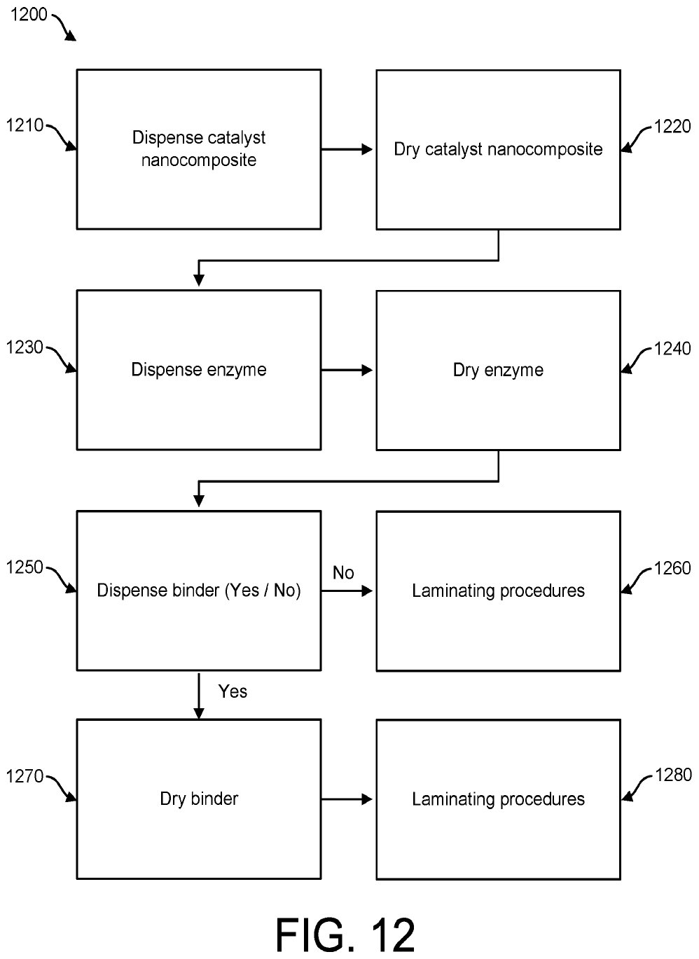

FIG. 12 is a process flow diagram 1200 illustration of the dispensing procedures of the nanocomposite, enzyme, and binder. Initially, at 1210, a catalyst nanocomposite is dispensed and then dried at 1220, thereafter, at 1230, the enzyme is dispensed and then dried at 1240. After that, at 1250, a binder can be dispensed, if so, the binder is dried at 1270 allowing to start the laminating procedures at 1260. If the binder is not dispensed the laminating procedures begins at 1260.

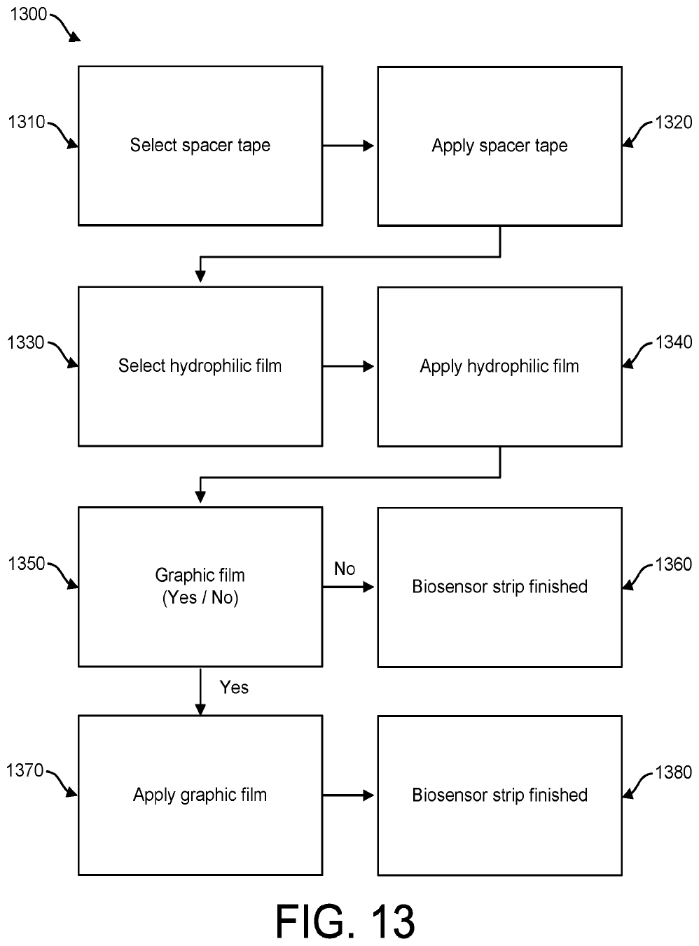

FIG. 13 is a process flow diagram 1300 illustration of the laminating procedures for assembling the biosensor. Initially, at 1310, the spacer tape is selected and applied at 1320, this is based on the inertness and thickness of the material. The thickness allows to define partially the volume of the capillary chamber. Thereafter, the hydrophilic film is selected at 1330, then applied at 1340. It is optional to add a graphic film at 1350, if so, the graphic film is applied at 1370 and teh, if not, the biosensor strip is finished at 1360 and 1380.

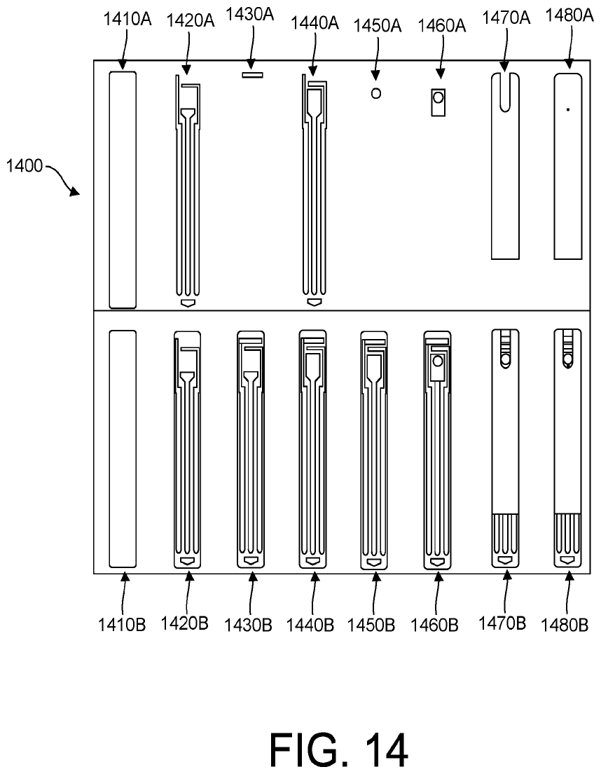

FIG. 14 is a flow process diagram 1400 illustrating various layers of the biosensor (1410A-1480A) and the corresponding stacked view (1410B-1480B) which correlates to a top view after the corresponding layer is applied (e.g., stacked view 1040B reflects all of layers 1010A-1040B). It will be appreciated that variations of the biosensor may include additional layers, different layers and/or omit certain layers depending on the desired design.

A first illustrated layer 1410A corresponds to a substrate. The second layer 1420A corresponds to a silver layer of the electrodes. The third layer 1430A corresponds to a screen-printed silver/silver chloride layer. The fourth layer 1440A corresponds to an inert conductive material screen printed layer. The fifth layer 1450A corresponds to the dispensing section, while 1460A to a dielectric screen-printed layer. The seventh layer 1470A corresponds to a spacer film. And the eight-layer 1480A corresponds to a hydrophilic film.

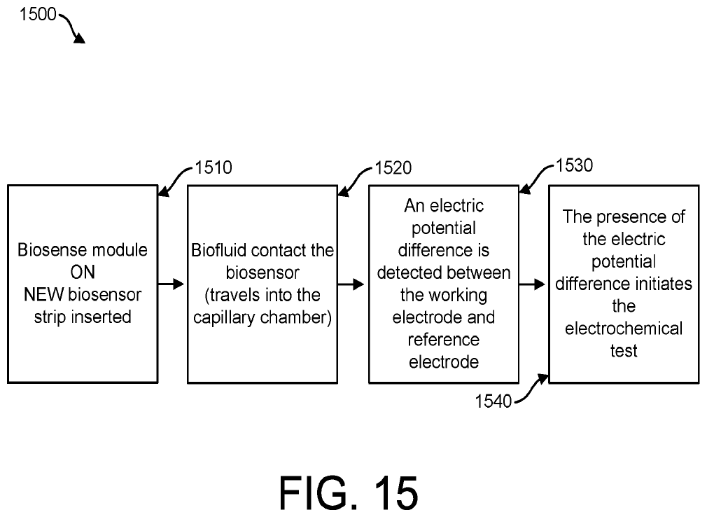

FIG. 15 is a flow process 1500 illustration relating the process of detecting the biofluid to initiate the electrochemical measurement. In the preferred embodiment of the biosensor, which is the strip like biosensor, the electronic module is monitoring the voltage in the strip. Initially, at 1510, while the Biosensor module is ON a new biosensor strip is connected. Thereafter, at 1520, the biofluid is in contact with the biosensor allowing to enter the biofluid into the capillary chamber. After that, at 1530, an electric potential is detected (>0V) between the counter and reference and finally the detection of such electric potential initiates the electrochemical test at 1540.

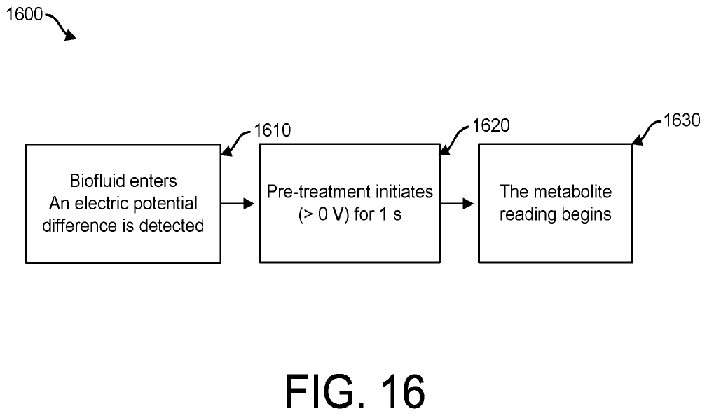

With reference to the process flow process diagram 1600 of FIG. 16, prior to performing the actual metabolite reading, a voltage is applied between the reference and working electrode. This procedure is called pre-treatment and it allows for the stabilization of the electroactive species and chemical compounds on the biosensor that might produce noise in the signal. The pre-treatment may or not be used depending on the manufacturing process of the biosensor. The voltage varies, between 0 and the voltage (e.g., the voltage bias) used for initiating the metabolite reading. Initially, at 1610, after detecting the biofluid, an electric potential is applied between working and reference electrode at 1620, thereafter, at 1630, the metabolite reading begins.

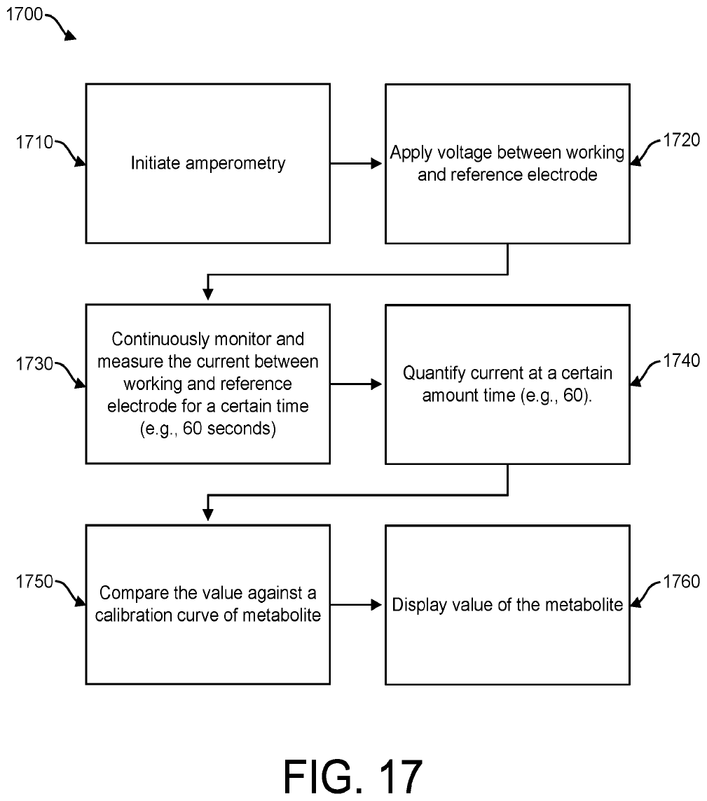

FIG. 17 is a flow process diagram 1700 of the process for initiating the metabolite reading. The process is two-fold at the molecular level, 1) oxidoreductase enzyme oxidases the metabolite of interest and hydrogen peroxide is produced 2) hydrogen peroxide is oxidized by applying a voltage between the working and reference electrodes. While the voltage is applied, simultaneously, the module is monitoring the current across the working and counter electrodes. This current is associated to the oxidation reaction of the hydrogen peroxide produced on the working electrode. In the biosensor preferred embodiment, the voltage applied may vary between 0.35 and 0.6 V and the procedure last between 5 to 60 seconds. As stated above, one molecule of hydrogen peroxide is produced from the oxidation of one molecule of metabolite of interest. Hence, the quantification of the metabolite is achievable by creating a respective calibration curve of the metabolite concentration VS current generated by the biosensor.

Initially, at 1710, the amperometry is initiated, thereafter, an at 1720 electric potential between and working electrode and reference electrode is applied for a certain amount of time (e.g., 60 seconds). At 1730, the electric current flow between counter electrode and working electrode is monitored and recorded (e.g., every 10 ms). After finishing the measurement, at 1740, the current detected is quantified at a certain given time (e.g., 60 seconds). At 1750, the value of current quantified is compared against a calibration curve allowing to determine the concentration of metabolite of interest and displayed at 1760.

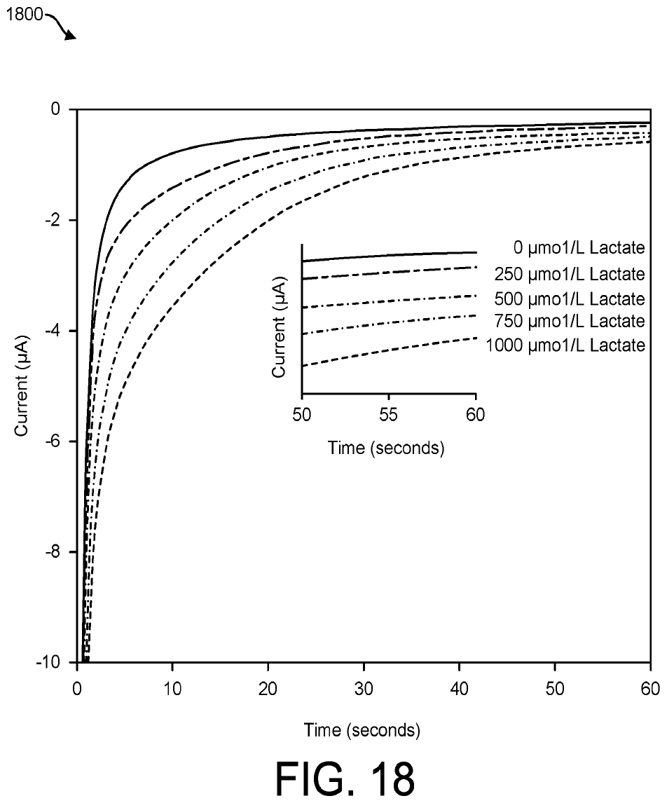

FIG. 18 is a graph 1800 illustrating the plotted current obtained by amperometry of the biosensor for 60 seconds when in contact with a biofluid conditioned with lactate at a certain concentration (0 to 1000 μmol/L). The inset graph shows the current response at the final stage of the amperometry which can be used to construct a calibration curve to determine the metabolite concentration presented in the sampled biofluid tested with the biosensor.

As noted above, the biosensor can be useful in a variety of applications. In one application, the biosensor acts as a lactate biosensor which, in turn, can be used for applications such as cardiac rehabilitation. Lactate tests require a blood sample which is impractical to perform during a prescribed workout routine. A non-invasive lactate test using the biosensors provided herein can be a significant simplification that would allow a cardiologist to monitor patients lactate levels dynamically during a workout. In practice, the patient rinses its mouth with water prior the workout to remove contaminants, solids and gel structures commonly found the mouth. Next, the clinician positions a swab inside the patients mouth for 30 seconds or more. Then, the saliva from the swab is filtered with a syringeless filter by pressing the head of the swab portion. The volume of saliva obtained depends on the quantity of adsorbed saliva with the swab that ranges from 10 to 40 microliters of volume.

By adding saliva filtrated to the biosensor, the biosensor is capable of detecting and quantify the amount of lactate in the biofluid sample, in this case saliva. This process can be done simply by inserting the sensor into the plunger, where the filtered saliva remains. The capillary chamber from the biosensor allows to enter the saliva and to have a fixed volume amount.

In some variations, saliva can be directly poured and filtered using a syringe and a syringe filter. Saliva/biofluid filtration can also be carried out using a vacuum pump such as piezoelectric device that applies negative pressure to suck out saliva from the mouth to be later filtered for the biosensor. A series of lactate saliva determinations can be done with the biosensor while performing a workout during cardiac rehabilitation. This procedure applies to a plurality of biofluids, such as the embodiment of lactate to estimate the lactate-threshold value.

The biosensors provided herein can also be used in monitoring glucose for patients having diabetes type 2. By measuring glucose from saliva non-invasively using the current biosensor, a user can more effectively manage their diabetes by monitoring glucose concentration in a semi-quantitative manner using a blood-glucose meter to periodically calibrate the biosensor and to record/evaluate blood-glucose trends. In other words, the biosensor can provide semiquantitative “blood-reference” values by performing a calibration measurement with a regular blood meter, for example, a glucose meter for diabetes can be used to perform a one-point or two-points calibration curve.

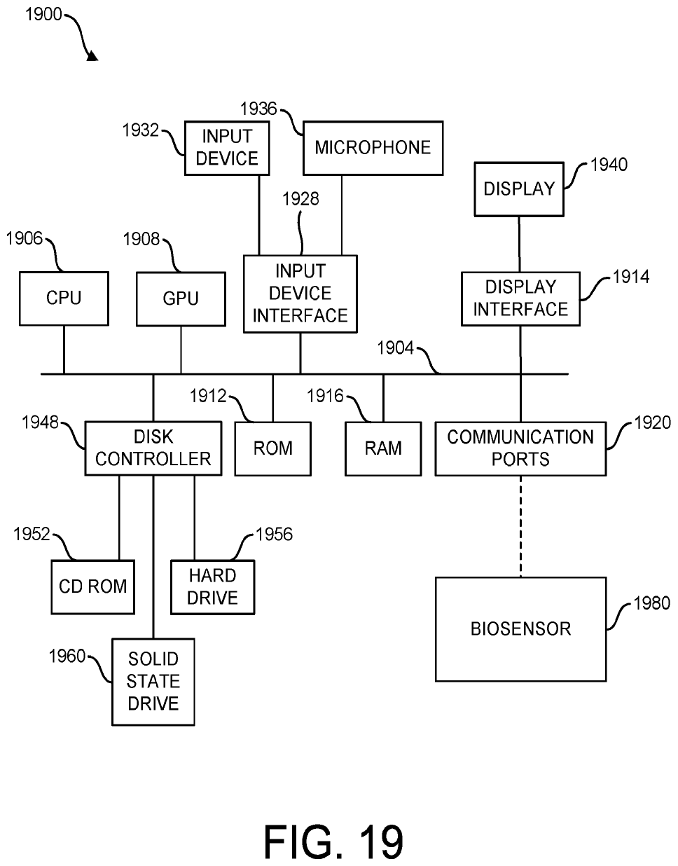

FIG. 19 is a diagram 1900 illustrating a sample computing device architecture for implementing various aspects described herein. A bus 1904 can serve as the information highway interconnecting the other illustrated components of the hardware. A processing system 1908 labeled CPU (central processing unit) (e.g., one or more computer processors/data processors at a given computer or at multiple computers), can perform calculations and logic operations required to execute a program. A non-transitory processor-readable storage medium, such as read only memory (ROM) 1912 and random-access memory (RAM) 1916, can be in communication with the processing system 1908 and can include one or more programming instructions for the operations specified here. Optionally, program instructions can be stored on a non-transitory computer-readable storage medium such as a magnetic disk, optical disk, recordable memory device, flash memory, or other physical storage medium.

In one example, a disk controller 1948 can interface with one or more optional disk drives to the system bus 1904. These disk drives can be external or internal floppy disk drives such as 1960, external or internal CD-R, CD-RW, DVD, or solid-state drives such as 1952, or external or internal hard drives 1956. As indicated previously, these various disk drives 1952, 1956, 1960 and disk controllers are optional devices. The system bus 1904 can also include at least one communication port 1920 to allow for communication with external devices either physically connected to the computing system or available externally through a wired or wireless network. In some cases, the at least one communication port 1920 includes or otherwise comprises a network interface.

To provide for interaction with a user, the subject matter described herein can be implemented on a computing device having a display device 1940 (e.g., a CRT (cathode ray tube) or LCD (liquid crystal display) monitor) for displaying information obtained from the bus 1904 via a display interface 1914 to the user and an input device 1932 such as keyboard and/or a pointing device (e.g., a mouse or a trackball) and/or a touchscreen by which the user can provide input to the computer. Other kinds of input devices 1932 can be used to provide for interaction with a user as well; for example, feedback provided to the user can be any form of sensory feedback (e.g., visual feedback, auditory feedback by way of a microphone 1936, or tactile feedback); and input from the user can be received in any form, including acoustic, speech, or tactile input. The input device 1932 and the microphone 1936 can be coupled to and convey information via the bus 1904 by way of an input device interface 1928. Other computing devices, such as dedicated servers, can omit one or more of the display 1940 and display interface 1914, the input device 1932, the microphone 1936, and input device interface 1928.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

1. A biosensor for monitoring one or more metabolites within a base fluid comprising:
a working electrode comprising:
an inert conductive layer;
a catalyst layer deposited over the inert conductive layer comprising a nanocomposite through which hydrogen peroxide may be adsorbed;
an enzyme layer deposited over the catalyst layer comprising one or more enzymes selected to perform a redox reaction towards one or more metabolites; and
a binder membrane providing supporting structure to the inert conductive layer, the catalyst layer, and the enzyme layer, the binder membrane selectively allowing molecules having a weight or size below a threshold from passing therethrough; a reference electrode to maintain a known and stable potential;
a counter electrode to allow current from or to the working electrode; and
an electrical interface in electrical communication with the inert conductive layer and configured to receive an electrical signal from the working electrode resulting from a biochemical reaction at or generated by one or more of the layers, the electrical signal corresponding to a level of the one or more metabolites in the base fluid.

2. The biosensor of claim 1, wherein the metabolite comprises one or more of: glucose, lactate, cholesterol, alcohol, fructose, pyruvate, D-amino acids, Acyl-CoA, or choline.

3. The biosensor of claim 1, wherein the base fluid is one or more of saliva, sweat, interstitial fluid, mucus, peritoneal fluid, feces, urine, tear fluid, capillary blood, or venous blood.

4. The biosensor of claim 1, wherein the one or more enzymes comprise lactate oxidase and the one or more metabolites comprise lactate.

5. The biosensor of claim 1, wherein the one or more enzymes comprise glucose oxidase and the one or more metabolites comprise glucose.

6. The biosensor of claim 1, wherein the binder membrane is composed by a selectively permeable porous protection layer on an outermost layer of the working electrode.

7. The biosensor of claim 1, wherein there are at least two electrodes having a corresponding enzyme layer which comprise different enzymes each detecting a different metabolite.

8. The biosensor of claim 1, wherein the binder membrane comprises one or more of: chitosan, chitosan deacetylated, fluoropolymer, fluoropolymer-copolymer with the ability of reacting with a cross-linking agent.

9. The biosensor of claim 1, wherein the catalyst layer comprises platinum nanoparticles.

10. The biosensor of claim 9, wherein the catalyst layer comprises carbon nanotubes.

11. The biosensor of claim 10, wherein the carbon nanotubes are chemically doped with dopant atoms.

12. The biosensor of claim 11, wherein the dopant atoms comprise one or more of nitrogen, boron, fluorine or hydrogen.

13. The biosensor of claim 9, wherein the platinum nanoparticles can be physically or chemically bonded to the supporting structure of the binder membrane and walls of the nanotubes.

14. A biosensor for monitoring one or more metabolites within a base fluid comprising:
one or more electrodes comprising:
a working electrode having a plurality of layers including:
a catalyst layer deposited over the inert conductive layer comprising a nanocomposite through which hydrogen peroxide may be adsorbed; and
an enzyme layer deposited over the catalyst layer comprising one or more enzymes selected to perform a redox reaction towards one or more metabolites; and
an electrical interface in electrical communication with the one or more electrodes configured to receive an electrical signal resulting from a biochemical reaction at or generated by one or more of the layers, the electrical signal corresponding to a level of the one or more metabolites in the base fluid.

15. The biosensor of claim 14, wherein the catalyst layer comprises carbon nanotubes.

16. The biosensor of claim 15, wherein the carbon nanotubes are chemically doped with dopant atoms.

17. The biosensor of claim 16, wherein the dopant atoms promote adsorption of molecular oxygen required by the one or more enzymes to perform catalysis of lactate to hydrogen peroxide.

18. The biosensor of claim 17, wherein the dopant atoms promote adsorption of hydrogen peroxide.

19. The biosensor of claim 14, wherein the one or more metabolites comprise lactate or glucose.

20. A biosensor for monitoring one or more metabolites within a base fluid comprising: a plurality of electrodes comprising:
a first working electrode having a plurality of layers including:
a catalyst layer deposited over the inert conductive layer comprising a nanocomposite through which hydrogen peroxide may be adsorbed; and
an enzyme layer deposited over the catalyst layer comprising one or more enzymes selected to perform a redox reaction towards a first metabolite;
a second working electrode having a plurality of layers including:
a catalyst layer deposited over the inert conductive layer comprising a nanocomposite through which hydrogen peroxide may be adsorbed; and
an enzyme layer deposited over the catalyst layer comprising one or more enzymes selected to perform a redox reaction towards a second metabolite different than the first metabolite; and
an electrical interface in electrical communication with the one or more electrodes configured to receive an electrical signal resulting from a biochemical reaction at or generated by one or more of the layers, the electrical signal corresponding to a level of the one or more metabolites in the base fluid.
